(19) United States
(12) Patent Application Publication
(10) Pub. No.: US 2007/0292922 A1
(43) Pub. Date: Dec. 20, 2007

(54) Regulated expression of recombinant proteins from adeno-associated viral vectors

(75) Inventors: Jianmin Fang, Palo Alto, CA (US); Karin Jooss, Bellevue, WA (US); Minh Nguyen, San Francisco, CA (US); Thomas C. Harding, San Francisco, CA (US); Timothy P. Clackson, Arlington, MA (US); Victor M. Rivera, Arlington, MA (US)

Correspondence Address: DLA PIPER US LLP, 153 TOWNSEND STREET, SUITE 800, SAN FRANCISCO, CA, 94107-1957, US

(73) Assignees: Cell Genesys, Inc., South San Francisco, CA (US); ARIAD Pharmaceuticals, Inc., Cambridge, MA (US)

(21) Appl. No.: 11/729,341

(22) Filed: Mar. 28, 2007

Related U.S. Application Data
(60) Provisional application No. 60/788,561, filed on Mar. 31, 2006.

(51) Int. Cl. C12P 21/08; C07K 16/00; C12N 15/00; C12N 5/10

(52) U.S. Cl. 435/69.6; 435/320.1; 435/328; 530/387.3

(57) ABSTRACT

Single AAV vector constructs for regulated expression of an immunoglobulin molecule or fragment thereof and methods of making and using the same are described. The AAV vectors comprise a regulated promoter operably linked to the coding sequence for a first and second immunoglobulin coding sequence, a sequence encoding a self-processing cleavage site between the coding sequence for the first and second immunoglobulin coding sequence and a additional proteolytic cleavage site, which provides a means to remove the self processing peptide sequence from an expressed immunoglobulin molecule or fragment thereof. The vector constructs find utility in enhanced production of biologically active immunoglobulins or fragments thereof in vitro and in vivo.

### CROSS-REFERENCE TO RELATED APPLICATIONS

This application claims the priority benefit of U.S. Provisional Patent Application No. 60/788,561, filed Mar. 31, 2006. The priority application is expressly incorporated by reference herein in its entirety.

### BACKGROUND OF THE INVENTION

1. Field of the Invention

The invention relates to novel adeno-associated viral (AAV) vector constructs that regulate the expression of recombinant full-length proteins or fragments thereof. The AAV constructs may be used for ex vivo or in vivo expression of a heterologous protein coding sequence by a cell or organ, or in vitro for the tightly regulated production of recombinant proteins by AAV vector-transduced cells.

2. Background of the Technology

Monoclonal antibodies have been proven as effective therapeutics for cancer and other diseases. Current antibody therapy often involves repeat administration and long term treatment regimens, which are associated with a number of disadvantages, such as inconsistent serum levels and limited duration of efficacy per administration such that frequent readministration is required and high cost. The use of antibodies as diagnostic tools and therapeutic modalities has found increasing use in recent years. The first FDA-approved monoclonal antibody for cancer treatment, Rituxan® (Rituximab) was approved in 1997 for the treatment of patients with non-Hodgkin's lymphoma and soon thereafter in 1998, Herceptin®, a humanized monoclonal antibody for treatment of patients with metastatic breast cancer, was approved. Numerous antibody-based therapies that are in various stages of clinical development are showing promise. One limitation to the widespread clinical application of antibody technology is that typically large amounts of antibody are required for therapeutic efficacy and the costs associated with production are significant. Chinese Hamster Ovarian (CHO) cells, SP20 and NSO2 myeloma cells are the most commonly used mammalian cell lines for commercial scale production of glycosylated human proteins such as antibodies. The yields obtained from mammalian cell line production typically range from 50-250 mg/L for 5-7 day culture in a batch fermentor or 300-1000 mg/L in 7-12 days in fed batch fermentors. High level production often relies upon gene amplification and selection of best performing clones which is time consuming and further increases the cost of development and production. In addition, stability issues with respect to antibody-producing cell lines are often evident following multiple passages.

There remains a need for improved systems for the regulated production of full length immunoglobulins and fragments thereof in vitro and in vivo for therapeutic use.

Adeno associated virus (AAV) is a preferred vector for delivering therapeutic genes due to its safety profile and capability of long term gene expression in vivo. Recombinant AAV vectors (rAAV) have been previously used to express single chain antibodies in vivo. Due to the limited transgene packaging capacity of AAV and its low transduction efficiency, it has been a technical challenge to have a tightly regulated system to express heavy and light chains of an antibody using a single AAV vector in order to generate full length antibodies.

The present invention addresses this need by demonstrating the feasibility and use of a novel approach for achieving regulated, high and consistent serum levels of full length antibodies following a single injection of a recombinant AAV vector.

### SUMMARY OF THE INVENTION

The present invention provides adeno-associated viral (AAV) vector constructs for the regulated expression of protein or polypeptide open reading frames from a single cell and methods of using the same.

In one preferred approach, the vectors have a rapalog-regulated promoter operably associated with a nucleotides sequence comprising a self-processing cleavage sequence and a proteolytic cleavage site between the protein or polypeptide coding sequences allowing for tightly regulated expression of more than one functional protein or polypeptide. The invention finds utility in production of two or more proteins or polypeptides or a protein or polypeptide having two or more domains (or chains) using an AAV vector where regulatable, sustainable expression occurs in a single cell. Exemplary AAV constructs comprise a rapalog-regulated hybrid ZFHD1/IL-2 promoter operably associated with a nucleotides sequence comprising a self-processing cleavage sequence and a proteolytic cleavage site for removal of the self-processing cleavage sequence from the expressed protein or polypeptide. The vector constructs find utility in methods relating to enhanced production of biologically active proteins, polypeptides or fragments thereof, in vitro and in vivo.

### BRIEF DESCRIPTION OF THE FIGURES

FIG. 1 is a schematic depiction of an AAV vector encoding transactivation regulatory elements for directing rapalog-regulated protein expression. The vector includes a rapamycin-binding p65-FRB fusion protein (Activation Domain) operatively linked to a liver-specific mouse transthyretin (mTTR) promoter and rapamycin-binding zinc finger fusion protein ZFHD1-2×FKBP (DNA Binding Domain) operatively linked to a minimal SV40 promoter.

FIG. 2 is a schematic depiction of an AAV expression cassette comprising a proteolytic cleavage site (Furin cleavage site; “F”) and a foot and mouth disease virus 2A self-processing site (2A) for expression of immunoglobulin heavy (H) and light (L) chains operatively linked to the rapamycin-regulated hybrid ZFHD1/IL-2 promoter. Rapamycin binds to each FK506 binding protein domain (FKBP) of the DNA Binding Domain to which two Activation Domains dimerize through the interaction of the large P13K homolog FRAP domain (FRB) and the bound rapamycin. The p65-activated transcription from the hybrid ZFHD1/IL-2 promoter occurs upon binding to ZFHDI sites in the promoter through the interactions with the zinc finger ZFHD1 domain.

FIG. 3 illustrates tightly regulated, rapamycin-dependent expression of a full-length rat anti-VEGFR2 monoclonal antibody (DC101 IgG1) in HuH7 cells following co-transfection with the AAV plasmids of FIGS. 1 and 2 in the absence or presence of 0.3, 1, 3, 10 or 3 nM rapamycin.

FIG. 4 illustrates rapamycin-dependent, in vivo expression of a full-length rat anti-VEGFR2 monoclonal antibody (DC101 IgG1). On Day 0, approximately 2.5×10¹¹ vp of each AAV vector shown in FIGS. 1 & 2 were co-administered i.v. to NCR nude mice followed by i.p. administration of 3 mg/kg body weight rapamycin or control vehicle on Days 21, 24, 28, 31, 35, and 38. Mice were bled on indicated days and the concentration of DC101 antibody present in serum samples (mcg/ml) at selected time points was determined by ELISA.

### DETAILED DESCRIPTION OF THE INVENTION

The present invention provides AAV viral vector constructs for regulated expression of recombinant immunoglobulin molecules or fragments thereof and methods for in vitro or in vivo use of the same. The vectors have a proteolytic cleavage site and a self-processing sequence between the heavy and light chain coding sequence of the immunoglobulin allowing for expression of a functional antibody molecule from a single expression cassette driven by a regulated promoter. Exemplary AAV vector constructs comprise a rapalog-regulated promoter operably associated with a sequence encoding a self-processing cleavage site between the heavy and light chain coding sequences of the immunoglobulin and further include a proteolytic cleavage site adjacent to the self-processing cleavage site for removal of amino acids derived from the self-processing cleavage site which remain following cleavage. The AAV vector constructs of the invention find utility in methods relating to regulated expression of full-length biologically active immunoglobulins or fragments thereof in vitro and in vivo.

The various compositions and methods of the invention are described below. Although particular compositions and methods are exemplified herein, it is understood that any of a number of alternative compositions and methods are applicable and suitable for use in practicing the invention. It will also be understood that an evaluation of the protein or polypeptide expression constructs (vectors) and methods of the invention may be carried out using procedures standard in the art.

Definitions

Unless otherwise indicated, all terms used herein have the same meaning as they would to one skilled in the art and the practice of the present invention will employ, unless otherwise indicated, conventional techniques of cell biology, molecular biology (including recombinant techniques), microbiology, biochemistry and immunology, which are known to those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991).

The term “vector”, as used herein, refers to a DNA or RNA molecule such as a plasmid, virus or other vehicle, which contains one or more heterologous or recombinant DNA sequences and is designed for transfer between different host cells. The terms “expression vector” and “gene therapy vector” refer to any vector that is effective to incorporate and express heterologous DNA fragments in a cell. A cloning or expression vector may comprise additional elements, for example, the expression vector may have two replication systems, thus allowing it to be maintained in two organisms, for example in human cells for expression and in a prokaryotic host for cloning and amplification. Any suitable vector can be employed that is effective for introduction of nucleic acids into cells such that protein or polypeptide expression results, e.g. a viral vector or non-viral plasmid vector. Any cells effective for expression, e.g., insect cells and eukaryotic cells such as yeast or mammalian cells are useful in practicing the invention.

The terms “heterologous DNA” and “heterologous RNA” refer to nucleotides that are not endogenous (native) to the cell or part of the genome in which they are present. Generally heterologous DNA or RNA is added to a cell by transduction, infection, transfection, transformation or the like, as further described below. Such nucleotides generally include at least one coding sequence, but the coding sequence need not be expressed. The term “heterologous DNA” may refer to a “heterologous coding sequence” or a “transgene”.

As used herein, the terms “protein” and “polypeptide” may be used interchangeably and typically refer to “proteins” and “polypeptides” of interest that are expresses using the self processing cleavage site-containing vectors of the present invention. Such “proteins” and “polypeptides” may be any protein or polypeptide useful for research, diagnostic or therapeutic purposes, as further described below.

The term “replication defective” as used herein relative to a viral gene therapy vector of the invention means the viral vector cannot independently further replicate and package its genome. For example, when a cell of a subject is infected with rAAV virions, the heterologous gene is expressed in the infected cells, however, due to the fact that the infected cells lack AAV rep and cap genes and accessory function genes, the rAAV is not able to replicate.

The term “operably linked” as used herein relative to a recombinant DNA construct or vector means nucleotide components of the recombinant DNA construct or vector are functionally related to one another for operative control of a selected coding sequence. Generally, “operably linked” DNA sequences are contiguous, and, in the case of a secretory leader, contiguous and in reading frame. However, enhancers do not have to be contiguous.

As used herein, the term “gene” or “coding sequence” means the nucleic acid sequence which is transcribed (DNA) and translated (mRNA) into a polypeptide in vitro or in vivo when operably linked to appropriate regulatory sequences. The gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, “the coding sequence for a first chain of an immunoglobulin molecule or a fragment thereof” refers to a nucleotide sequence encoding a protein molecule including, but not limited to a light chain or heavy chain for an antibody or immunoglobulin, or a fragment thereof.

As used herein, “the coding sequence for a second chain of an immunoglobulin molecule or a fragment thereof” refers to a nucleotide sequence encoding a protein molecule including, but not limited to a light chain or heavy chain for an antibody or immunoglobulin, or a fragment thereof.

A “promoter” is a DNA sequence that directs the binding of RNA polymerase and thereby promotes RNA synthesis, i.e., a minimal sequence sufficient to direct transcription. Promoters and corresponding protein or polypeptide expression may be cell-type specific, tissue-specific, or species specific. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

“Enhancers” are cis-acting elements that stimulate or inhibit transcription of adjacent genes. An enhancer that inhibits transcription also is termed a “silencer”. Enhancers can function (i.e., can be associated with a coding sequence) in either orientation, over distances of up to several kilobase pairs (kb) from the coding sequence and from a position downstream of a transcribed region.

A “regulatable promoter” is any promoter whose activity is affected by a cis or trans acting factor (e.g., an inducible promoter, such as an external signal or agent).

“Rapamycin” is a macrolide antibiotic produced by Streptomyces hygroscopicus which binds to a FK506-binding protein, FKBP, with high affinity to form a rapamycin:FKBP complex. The rapamycin:FKBP complex binds with high affinity to the large cellular protein, FRAP, to form an FKBP/rapamycin complex with FRAP. Rapamycin acts as a dimerizer or adapter to join FKBP to FRAP.

As used herein, the term “rapalog” is meant to include structural variants of rapamycin including analogs, homologs, derivatives and other compounds related structurally to rapamycin. Such structural variants include modifications such as demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring. See, e.g., U.S. Pat. Nos. 6,187,757; 5,525,610; 5,310,903 and 5,362,718, expressly incorporated by reference herein. Exemplary rapalogs include, but are not limited to rapamycin (sirolimus), temsirolimus, everolimus, ABT578, AP23573 and biolimus.

A “rapamycin-regulated promoter” refers to a promoter the activity of which is regulated by the presence or absence of rapamycin.

The terms “transcriptional regulatory protein”, “transcriptional regulatory factor” and “transcription factor” are used interchangeably herein, and refer to a nuclear protein that binds a DNA response element and thereby transcriptionally regulates the expression of an associated gene or genes. Transcriptional regulatory proteins generally bind directly to a DNA response element, however in some cases binding to DNA may be indirect by way of binding to another protein that in turn binds to, or is bound to a DNA response element.

As used herein, the term “sequence identity” means nucleic acid or amino acid sequence identity between two or more aligned sequences, when aligned using a sequence alignment program. The terms “% homology” and “% identity” are used interchangeably herein and refer to the level of nucleic acid or amino acid sequence identity between two or more aligned sequences, when aligned using a sequence alignment program. For example, 80% homology means the same thing as 80% sequence identity determined by a defined algorithm under defined conditions.

The terms “identical” or percent “identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the sequence comparison algorithms described herein, e.g. the Smith-Waterman algorithm, or by visual inspection.

A “self-processing cleavage site” or “self-processing cleavage sequence” is defined herein as a post-translational or co-translational processing cleavage site or sequence. Such a “self-processing cleavage” site or sequence refers to a DNA or amino acid sequence, exemplified herein by a 2A site, sequence or domain or a 2A-like site, sequence or domain. As used herein, a “self-processing peptide” is defined herein as the peptide expression product of the DNA sequence that encodes a self-processing cleavage site, sequence or domain, which during translation mediates rapid intramolecular (cis) cleavage of a protein or polypeptide comprising the self-processing cleavage site to yield discrete mature protein or polypeptide products.

As used herein, the term “additional proteolytic cleavage site”, refers to a sequence which is incorporated into an expression construct of the invention adjacent a self-processing cleavage site, such as a 2A or 2A like sequence, and provides a means to remove additional amino acids that remain following cleavage by the self processing cleavage sequence. Exemplary “additional proteolytic cleavage sites” are described herein and include, but are not limited to, furin cleavage sites with the consensus sequence RXK(R)R (SEQ ID NO: 10). Such furin cleavage sites can be cleaved by endogenous subtilisin-like proteases, such as furin and other serine proteases within the protein secretion pathway.

As used herein, the terms “immunoglobulin” and “antibody” may be used interchangeably and refer to intact immunoglobulin or antibody molecules as well as fragments thereof, such as Fa, F (ab′)2, and Fv, which are capable of binding an antigenic determinant. Such an “immunoglobulin” and “antibody” is composed of two identical light polypeptide chains of molecular weight approximately 23,000 daltons, and two identical heavy chains of molecular weight 53,000-70,000. The four chains are joined by disulfide bonds in a “Y” configuration. Heavy chains are classified as gamma (IgG), mu (IgM), alpha (IgA), delta (IgD) or epsilon (IgE) and are the basis for the class designations of immunoglobulins, which determines the effector function of a given antibody. Light chains are classified-as either kappa or lambda. When reference is made herein to an “immunoglobulin or fragment thereof”, it will be understood that such a “fragment thereof” is an immunologically functional immunoglobulin fragment.

The term “humanized antibody” refers to an antibody molecule in which one or more amino acids of the antigen binding regions of a non-human antibody have been replaced in order to more closely resemble a human antibody, while retaining the binding activity of the original non-human antibody. See, e.g., U.S. Pat. No. 6,602,503.

The term “antigenic determinant”, as used herein, refers to that fragment of a molecule (i.e., an epitope) that makes contact with a particular antibody. Numerous regions of a protein or fragment of a protein may induce the production of antibodies that binds specifically to a given region of the three-dimensional structure of the protein. These regions or structures are referred to as antigenic determinants. An antigenic determinant may compete with the intact antigen (i.e., the immunogen used to elicit the immune response) for binding to an antibody.

The term “fragment,” when referring to a recombinant protein or polypeptide of the invention means a polypeptide which has an amino acid sequence which is the same as part of, but not all of, the amino acid sequence of the corresponding full length protein or polypeptide, and which retains at least one of the functions or activities of the corresponding full length protein or polypeptide. The fragment preferably includes at least 20-100 contiguous amino acid residues of the full-length protein or polypeptide.

The terms “administering” or “introducing”, as used herein refer to delivery of a vector for recombinant protein expression to a cell or to cells and/or organs of a subject. Such administering or introducing may take place in vivo, in vitro or ex vivo. A vector for recombinant protein or polypeptide expression may be introduced into a cell by transfection, which typically means insertion of heterologous DNA into a cell by physical means (e.g., calcium phosphate transfection, electroporation, microinjection or lipofection); infection, which typically refers to introduction by way of an infectious agent, i.e. a virus; or transduction, which typically means stable infection of a cell with a virus or the transfer of genetic material from one microorganism to another by way of a viral agent (e.g., a bacteriophage).

“Transformation” is typically used to refer to bacteria comprising heterologous DNA or cells that express an oncogene and have therefore been converted into a continuous growth mode such as tumor cells. A vector used to “transform” a cell may be a plasmid, virus or other vehicle.

Typically, a cell is referred to as “transduced”, “infected”, “transfected” or “transformed” dependent on the means used for administration, introduction or insertion of heterologous DNA (i.e., the vector) into the cell. The terms “transduced”, “transfected” and “transformed” may be used interchangeably herein regardless of the method of introduction of heterologous DNA. A cell may be “transduced” by infection with a viral vector.

As used herein, the terms “stably transformed”, “stably transfected” and “transgenic” refer to cells that have a non-native (heterologous) nucleic acid sequence integrated into the genome. Stable transfection is demonstrated by the establishment of cell lines or clones comprised of a population of daughter cells containing the transfected DNA stably integrated into their genomes. In some cases, “transfection” is not stable, i.e., it is transient. In the case of transient transfection, the exogenous or heterologous DNA is expressed, however, the introduced sequence is not integrated into the genome and is considered to be episomal.

As used herein, “ex vivo administration” refers to a process where primary cells are taken from a subject, a vector is administered to the cells to produce transduced, infected or transfected recombinant cells and the recombinant cells are readministered to the same or a different subject.

A “multicistronic transcript” refers to an mRNA molecule that contains more than one protein coding region, or cistron. A mRNA comprising two coding regions is denoted a “bicistronic transcript.” The “5′-proximal” coding region or cistron is the coding region whose translation initiation codon (usually AUG) is closest to the 5′-end of a multicistronic mRNA molecule. A “5′-distal” coding region or cistron is one whose translation initiation codon (usually AUG) is not the closest initiation codon to the 5′ end of the mRNA. The terms “5′-distal” and “downstream” are used synonymously to refer to coding regions that are not adjacent to the 5′ end of a mRNA molecule.

As used herein, “co-transcribed” means that two (or more) coding regions or polynucleotides are under transcriptional control of a single transcriptional control or regulatory element.

As used herein, a “therapeutic” gene refers to a gene that, when expressed, confers a beneficial effect on the cell or tissue in which it is present, or on a mammal in which the gene is expressed. Examples of beneficial effects include amelioration of a sign or symptom of a condition or disease, prevention or inhibition of a condition or disease, or conferral of a desired characteristic. Therapeutic genes include genes that correct a genetic deficiency in a cell or mammal.

The term “host cell”, as used herein refers to a cell that has been transduced, infected, transfected or transformed with a vector. The vector may be a plasmid, a viral particle, a phage, etc. The culture conditions, such as temperature, pH and the like, are those previously used with the host cell selected for expression, and will be apparent to those skilled in the art. It will be appreciated that the term “host cell” refers to the original transduced, infected, transfected or transformed cell and progeny thereof.

The term “expression” refers to the transcription and/or translation of an endogenous gene, transgene or coding region in a cell. In the case of an antisense construct, expression may refer to the transcription of the antisense DNA only.

As used herein, the terms “biological activity” and “biologically active”, refer to the activity attributed to a particular protein in a cell line in culture or in vivo. The “biological activity” of an “immunoglobulin”, “antibody” or fragment thereof refers to the ability to bind an antigenic determinant and thereby facilitate immunological function.

As used herein, the terms “tumor” and “cancer” refer to a cell that exhibits a loss of growth control and forms unusually large clones of cells. Tumor or cancer cells generally have lost contact inhibition and may be invasive and/or have the ability to metastasize.

Immunoglobulins and Fragments Thereof

Antibodies are immunoblobulin proteins that are heterodimers of a heavy and light chain and have proven difficult to express in a full length form from a single vector in mammalian culture expression systems. Three methods are currently used for production of vertebrate antibodies, in vivo immunization of animals to produce “polyclonal” antibodies, in vitro cell culture of B-cell hybridomas to produce monoclonal antibodies (Kohler, et al., Eur. J. Immunol., 6: 511, 1976; Antibodies: A Laboratory Manual, Cold Spring Harbor Laboratory, 1988; incorporated by reference herein) and recombinant DNA technology (described for example in Cabilly et al., U.S. Pat. No. 6,331,415, incorporated by reference herein).

The basic molecular structure of immunoglobulin polypeptides is well known to include two identical light chains with a molecular weight of approximately 23,000 daltons, and two identical heavy chains with a molecular weight 53,000-70,000, where the four chains are joined by disulfide bonds in a “Y” configuration. The amino acid sequence runs from the N-terminal end at the top of the Y to the C-terminal end at the bottom of each chain. At the N-terminal end is a variable region (of approximately 100 amino acids in length) which provides for the specificity of antigen binding.

The present invention is directed to improved methods for production of immunoglobulins of all types, including, but not limited to full length antibodies and antibody fragments having a native sequence (i.e. that sequence produced in response to stimulation by an antigen), single chain antibodies which combine the antigen binding variable region of both the heavy and light chains in a single stably-folded polypeptide chain; univalent antibodies (which comprise a heavy chain/light chain dimer bound to the Fc region of a second heavy chain); “Fab fragments” which include the full “Y” region of the immunoglobulin molecule, i.e., the branches of the “Y”, either the light chain or heavy chain alone, or portions, thereof (i.e., aggregates of one heavy and one light chain, commonly known as Fab′); “hybrid immunoglobulins” which have specificity for two or more different antigens (e.g., quadromas or bispecific antibodies as described for example in U.S. Pat. No. 6,623,940); “composite immunoglobulins” wherein the heavy and light chains mimic those from different species or specificities; and “chimeric antibodies” wherein portions of each of the amino acid sequences of the heavy and light chain are derived from more than one species (i.e., the variable region is derived from one source such as a murine antibody, while the constant region is derived from another, such as a human antibody).

The compositions and methods of the invention find utility in production of immunoglobulins or fragments thereof wherein the heavy or light chain is “mammalian”, “chimeric” or modified in a manner to enhance its efficacy. Modified antibodies include both amino acid and nucleotide sequence variants which retain the same biological activity of the unmodified form and those which are modified such that the activity is altered, i.e., changes in the constant region that improve complement fixation, interaction with membranes, and other effector functions, or changes in the variable region that improve antigen binding characteristics. The compositions and methods of the invention further include catalytic immunoglobulins or fragments thereof.

A “variant” immunoglobulin-encoding polynucleotide sequence may encode a “variant” immunoglobulin amino acid sequence which is altered by one or more amino acids from the reference polypeptide sequence. The variant polynucleotide sequence may encode a variant amino acid sequence which contains “conservative” substitutions, wherein the substituted amino acid has structural or chemical properties similar to the amino acid which it replaces. In addition, or alternatively, the variant polynucleotide sequence may encode a variant amino acid sequence which contains “non-conservative” substitutions, wherein the substituted amino acid has dissimilar structural or chemical properties to the amino acid which it replaces. Variant immunoglobulin-encoding polynucleotides may also encode variant amino acid sequences which contain amino acid insertions or deletions, or both. Furthermore, a variant “immunoglobulin-encoding polynucleotide may encode the same polypeptide as the reference polynucleotide sequence but, due to the degeneracy of the genetic code, has a polynucleotide sequence which is altered by one or more bases from the reference polynucleotide sequence.

The term “fragment,” when referring to a recombinant immunoglobulin of the invention means a polypeptide which has an amino acid sequence which is the same as part of but not all of the amino acid sequence of the corresponding full length immunoglobulin protein, which either retains essentially the same biological function or activity as the corresponding full length protein, or retains at least one of the functions or activities of the corresponding full length protein. The fragment preferably includes at least 20-100 contiguous amino acid residues of the full length immunoglobulin.

The potential of antibodies as therapeutic modalities is currently limited by the production capacity and excessive cost of the current technology. The single rAAV vector immunoblobulin expression system of the invention permits the expression and delivery of two or more coding sequences, i.e., immunoglobulins with bi- or multiple-specificities from a single AAV vector. The present invention addresses the limitations in the prior art and is applicable to any immunoglobulin (i.e. an antibody) or fragment thereof as further detailed herein, including engineered antibodies, e.g., single chain antibodies, full-length antibodies or antibody fragments.

The invention relies on the expression of immunoglobulin heavy and light chains using a single regulated promoter wherein the heavy and light chains are expressed in substantially equal ratios. The linking of proteins in the form of polyproteins is a strategy adopted in the replication of many viruses including picomaviridae. Upon translation, virus-encoded self-processing peptides mediate rapid intramolecular (cis) cleavage of the polyprotein to yield discrete mature protein products and subsequent cleavage at the proteolytic cleavage site removes the majority of the remaining self-processing sequence. The present invention provides advantages over the use of an IRES in that a vector for recombinant immunoglobulin expression comprising a self-processing peptide (exemplified herein by 2A peptides) is provided which facilitates expression of immunoglobulin heavy and light chain coding sequences using a single regulated promoter, wherein the immunoglobulin heavy and light chain coding sequences are expressed in a substantially equimolar ratio. The expression of heavy and light chains in substantially equal molar ratios may be demonstrated, for example, by Western blot analysis, where the heavy and light chain proteins are separated by SDS-PAGE under reducing conditions, probed using an anti-rat or anti-human IgG polyclonal antibody and visualized using commercially available kits according to the manufacturer's instructions.

Self-Processing Cleavage Sites or Sequences

A “self-processing cleavage site” or “self-processing cleavage sequence” as defined above refers to a DNA or amino acid sequence, wherein upon translation, rapid intramolecular (cis) cleavage of a polypeptide comprising the self-processing cleavage site occurs to yield discrete mature protein products. Such a “self-processing cleavage site”, may also be referred to as a post-translational or co-translational processing cleavage site, exemplified herein by a 2A site, sequence or domain. A 2A site, sequence or domain demonstrates a translational effect by modifying the activity of the ribosome to promote hydrolysis of an ester linkage, thereby releasing the polypeptide from the translational complex in a manner that allows the synthesis of a discrete downstream translation product to proceed (Donnelly, 2001). Alternatively, a 2A site or domain demonstrates “auto-proteolysis” or “cleavage” by cleaving its own C-terminus in cis to produce primary cleavage products (Furler; Palmenberg, Ann. Rev. Microbiol. 44:603-623 (1990)).

Although the mechanism is not part of the invention, the activity of 2A may involve ribosomal skipping between codons which prevents formation of peptide bonds (de Felipe et al., Human Gene Therapy 11:1921-1931 (2000); Donnelly et al., J. Gen. Virol. 82:1013-1025 (2001); although it has been considered that the domain acts more like an autolytic enzyme (Ryan et al., Virol. 173:35-45 (1989)). Studies in which the Foot and Mouth Disease Virus (FMDV) 2A coding region was cloned into expression vectors and transfected into target cells have established that FMDV 2A cleavage of artificial reporter polyproteins is efficient in a broad range of heterologous expression systems (wheat-germ lysate and transgenic tobacco plant (Halpin et al., U.S. Pat. No. 5,846,767 (1998) and Halpin et al., The Plant Journal 17:453-459 (1999)); Hs 683 human glioma cell line (de Felipe et al., Gene Therapy 6:198-208 (1999); hereinafter referred to as “de Felipe II”); rabbit reticulocyte lysate and human HTK-143 cells (Ryan et al., EMBO J. 13:928-933 (1994)); and insect cells (Roosien et al., J. Gen. Virol. 71:1703-1711 (1990)). FMDV 2A-mediated cleavage of a heterologous polyprotein has been shown for IL-12 (p40/p35 heterodimer; Chaplin et al., J. Interferon Cytokine Res. 19:235-241 (1999)). In transfected COS-7 cells, FMDV 2A mediated the cleavage of a p40-2A-p35 polyprotein into biologically functional subunits p40 and p35 having activities associated with IL-12.

The FMDV 2A sequence has been incorporated into retroviral vectors, alone or combined with different IRES sequences to construct bicistronic, tricistronic and tetracistronic vectors. The efficiency of 2A-mediated gene expression in animals was demonstrated by Furler (2001) using recombinant adeno-associated viral (AAV) vectors encoding a-synuclein and EGFP or Cu/Zn superoxide dismutase (SOD-1) and EGFP linked via the FMDV 2A sequence. EGFP and a-synuclein were expressed at substantially higher levels from vectors which included a 2A sequence relative to corresponding IRES-based vectors, while SOD-1 was expressed at comparable or slightly higher levels. Furler also demonstrated that the 2A sequence results in bicistronic gene expression in vivo after injection of 2A-containing AAV vectors into rat substantia nigra. Recently, 2A peptides and 2A-like sequences were demonstrated to be effective in efficient translation of four cistrons using a retroviral vector (Szymczak A L et al., Nat Biotechnol. 2004 May 22(5):589-94).

For the present invention, the DNA sequence encoding a self-processing cleavage site is exemplified by viral sequences derived from a picornavirus, including but not limited to an entero-, rhino-, cardio-, aphtho- or Foot-and-Mouth Disease Virus (FMDV). In a preferred embodiment, the self-processing cleavage site coding sequence is derived from a FMDV. Self-processing cleavage sites include but are not limited to 2A and 2A-like domains (Donnelly et al., J. Gen. Virol. 82:1027-1041 (2001), expressly incorporated by reference in its entirety.

Positional subcloning of a 2A sequence between two or more heterologous DNA sequences for the inventive vector construct allows the delivery and expression of two or more genes through a single expression vector. Preferably, self processing cleavage sites such as FMDV 2A sequences provide a unique means to express and deliver from a single viral vector, two or multiple proteins, polypeptides or peptides which can be individual parts of, for example, an antibody, heterodimeric receptor or heterodimeric protein.

FMDV 2A is a polyprotein region which functions in the FMDV genome to direct a single cleavage at its own C-terminus, thus functioning in cis. The FMDV 2A domain is typically reported to be about nineteen amino acids in length (LLNFDLLKLAGDVESNPGP; SEQ ID NO: 1); (TLNFDLLKLAGDVESNPGP; SEQ ID NO: 2; Ryan et al., J. Gen. Virol. 72:2727-2732 (1991)), however oligopeptides of as few as fourteen amino acid residues (LLKLAGDVESNPGP; SEQ ID NO: 3) have been shown to mediate cleavage at the 2A C-terminus in a fashion similar to its role in the native FMDV polyprotein processing.

Variations of the 2A sequence have been studied for their ability to mediate efficient processing of polyproteins (Donnelly ML et al. 2001). Homologues and variants of a 2A sequence are included within the scope of the invention and include but are not limited to the sequences presented in Table 1, below:

TABLE 1
Table of Exemplary 2A Sequences

(SEQ ID NO: 1)

LLNFDLLKLAGDVESNPGP

(SEQ ID NO: 2)

TLNFDLLKLAGDVESNPGP;

(SEQ ID NO: 3)

LLKLAGDVESNPGP

(SEQ ID NO: 4)

NFDLLKLAGDVESNPGP

(SEQ ID NO: 5)

QLLNFDLLKLAGDVESNPGP

(SEQ ID NO: 6)

APVKQTLNFDLLKLAGDVESNPGP.

(SEQ ID NO: 7)

VTELLYRMKRAETYCPRPLLAIHPTEARHKQKIVAPVKQTLNFDLLKLAG
DVESNPGP

(SEQ ID NO: 8)

LLAIHPTEARHKQKIVAPVKQTLNFDLLKLAGDVESNPGP

(SEQ ID NO: 9)

EARHKQKIVAPVKQTLNFDLLKLAGDVESNPGP

Distinct advantages of 2A sequences and variants thereof are their use in facilitating self-processing of polyproteins. This invention includes any vector (plasmid or viral based) which includes the coding sequence for proteins or polypeptides linked via self-processing cleavage sites such that the individual proteins are expressed in equimolar or close to equimolar amounts following the cleavage of the polyprotein due to the presence of the self-processing cleavage site, e.g., a 2A domain. These proteins may be heterologous to the vector itself, to each other or to the self-processing cleavage site, e.g., FMDV.

The small size of the 2A coding sequence further enables its use in vectors with a limited packaging capacity for a coding sequence such as AAV. The utility of AAV vectors can be further expanded since the 2A sequence eliminates the need for dual promoters. The expression level of individual proteins, polypeptides or peptides from a promoter driving a single open reading frame comprising more than two coding sequences in conjunction with 2A are closer to equimolar as compared to the expression level achievable using IRES sequences or dual promoters. Elimination of dual promoters also reduces promoter interference that may result in reduced and/or impaired levels of expression for each coding sequence.

In one preferred embodiment, the FMDV 2A sequence included in a vector according to the invention encodes amino acid residues comprising LLNFDLLKLAGDVESNPGP (SEQ ID NO:1). Alternatively, a vector according to the invention may encode amino acid residues for other 2A-like regions as discussed in Donnelly et al., J. Gen. Virol. 82:1027-1041 (2001) and including but not limited to a 2A-like domain from picornavirus, insect virus, Type C rotavirus, trypanosome repeated sequences or the bacterium, Thermatoga maritima.

The invention contemplates use of nucleotide sequence variants that encode a 2A or 2A-like polypeptide, such as a nucleic acid coding sequence for a 2A or 2A-like polypeptide which has a different codon for one or more of the amino acids relative to that of the parent nucleotide. Such variants are specifically contemplated and encompassed by the present invention. Sequence variants of 2A peptides and polypeptides are included within the scope of the invention as well.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), by the BLAST algorithm, Altschul et al., J Mol. Biol. 215: 403-410 (1990), with software that is publicly available through the National Center for Biotechnology Information (http://www.ncbi.nlm.nih.gov/), or by visual inspection (see generally, Ausubel et al., infra). For purposes of the present invention, optimal alignment of sequences for comparison is most preferably conducted by the local homology algorithm of Smith & Waterman, Adv. Appl. Math. 2: 482 (1981). See, also, Altschul, S. F. et al., 1990 and Altschul, S. F. et al., 1997.

In accordance with the present invention, also encompassed are sequence variants which encode self-processing cleavage polypeptides and polypeptides themselves that have 80, 85, 88, 89, 90, 91, 92, 93, 94, 95, 96, 97, 98, 99% or more sequence identity to the native sequence.

A nucleotide sequence is considered to be “selectively hybridizable” to a reference nucleotide sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm-5° C. (5° below the Tm of the probe); “high stringency” at about 5-10° below the Tm; “intermediate stringency” at about 10-20° below the Tm of the probe; and “low stringency” at about 20-25° below the Tm. Functionally, maximum stringency conditions may be used to identify sequences having strict identity or near-strict with the hybridization probe; while high stringency conditions are used to identify sequences having about 80% or more sequence identity with the probe.

Moderate and high stringency hybridization conditions are well known in the art (see, for example, Sambrook, et al, 1989, Chapters 9 and 11, and in Ausubel, F. M., et al., 1993. An example of high stringency conditions includes hybridization at about 42° C. in 50% formamide, 5×SSC, 5× Denhardt's solution, 0.5% SDS and 100 mg/ml denatured carrier DNA followed by washing two times in 2×SSC and 0.5% SDS at room temperature and two additional times in 0.1×SSC and 0.5% SDS at 42° C. 2A sequence variants that encode a polypeptide with the same biological activity as the 2A polypeptides described herein and hybridize under moderate to high stringency hybridization conditions are considered to be within the scope of the present invention.

As a result of the degeneracy of the genetic code, a number of coding sequences can be produced which encode the same 2A or 2A-like polypeptide. For example, the triplet CGT encodes the amino acid arginine. Arginine is alternatively encoded by CGA, CGC, CGG, AGA, and AGG. Therefore it is appreciated that such substitutions in the coding region fall within the sequence variants that are covered by the present invention.

It is further appreciated that such sequence variants may or may not hybridize to the parent sequence under conditions of high stringency. This would be possible, for example, when the sequence variant includes a different codon for each of the amino acids encoded by the parent nucleotide. Such variants are, nonetheless, specifically contemplated and encompassed by the present invention.

Removal of Self-Processing Cleavage Peptide Sequences

One concern associated with the use of self-processing peptides, such as 2A or 2A-like sequences is that the N terminus of the first polypeptide contains amino acids derived from the self-processing peptide, i.e. 2A-derived amino acid residues. These amino acid residues are “foreign” to the host and may elicit an immune response when the recombinant protein is expressed or delivered in vivo (i.e., expressed from a viral or non-viral vector in the context of gene therapy or administered as an in vitro-produced recombinant protein). In addition, if not removed, 2A-derived amino acid residues may interfere with protein secretion in producer cells and/or alter protein conformation, resulting in a less than optimal expression level and/or reduced biological activity of the recombinant protein. The invention includes gene expression constructs, engineered such that a proteolytic cleavage site is provided between a polypeptide coding sequence and the self processing cleavage site (i.e., a 2A-sequence) as a means for removal of remaining self processing cleavage site derived amino acid residues following cleavage.

Examples of proteolytic cleavage sites are furin cleavage sites with the consensus sequence RXK(R)R (SEQ ID NO: 10), which can be cleaved by endogenous subtilisin-like proteases, such as furin and other serine proteases within the protein secretion pathway. As shown in U.S. Ser. No. 10/831302, expressly incorporated by reference herein, the inventors have demonstrated that 2A residues at the N terminus of the first protein can be efficiently removed by introducing a furin cleavage site RAKR (SEQ ID NO:11) between the first polypeptide and the 2A sequence. In addition, use of a plasmid containing a nucleotide sequence encoding a 2A sequence and a furin cleavage site adjacent to the 2A site was shown to result in a higher level of protein expression than a plasmid containing the 2A sequence alone. This improvement provides a further advantage in that when 2A residues are removed from the N-terminus of the protein, longer 2A- or 2A like sequences or other self-processing sequences can be used. Such longer self-processing sequences such as 2A- or 2A like sequences may facilitate better equimolar expression of two or more polypeptides by way of a single promoter.

It is advantageous to employ antibodies or analogues thereof with fully human characteristics. These reagents avoid the undesired immune responses induced by antibodies or analogues originating from non-human species. To address possible host immune responses to amino acid residues derived from self-processing peptides, the coding sequence for a proteolytic cleavage site may be inserted (using standard methodology known in the art) between the coding sequence for the first protein and the coding sequence for the self-processing peptide so as to remove the self-processing peptide sequence from the expressed polypeptide, i.e. the antibody. This finds particular utility in therapeutic or diagnostic antibodies for use in vivo.

Any additional proteolytic cleavage site known in the art which can be expressed using recombinant DNA technology vectors may be employed in practicing the invention. Exemplary additional proteolytic cleavage sites which can be inserted between a polypeptide or protein coding sequence and a self processing cleavage sequence (such as a 2A sequence) include, but are not limited to a:

a).

(SEQ ID NO: 10)

Furin cleavage site: RXK(R)R;
b).

(SEQ ID NO: 12)

Factor Xa cleavage site: IE(D)GR;
c).

(SEQ ID NO: 13)

Signal peptidase I cleavage site: e.g. LAGFATVAQA;
and
d).

(SEQ ID NO: 14)

Thrombin cleavage site: LVPRGS.

As detailed herein, the 2A peptide sequence provides a “cleavage” side that facilitates the generation of both chains of an immunoglobulin or other protein during the translation process. In one exemplary embodiment, the C-terminus of the first protein, for example the immunoglobulin heavy chain, contains approximately 13 amino acid residues which are derived from the 2A sequence itself. The number of residual amino acids is dependent upon the 2A sequence used. As set forth above and shown in the Examples, when a furin cleavage site sequence, e.g., RAKR, is inserted between the first protein and the 2A sequence, the 2A residues are removed from the C-terminus of the first protein. However, mass spectrum data indicates that the C-terminus of the first protein expressed from the RAKR-2A construct contains two additional amino acid residues, RA, derived from the furin cleavage site RAKR.

In one embodiment, the invention provides a method for removal of these residual amino acids and a composition for expression of the same. A number of novel constructs have been designed that provide for removal of these additional amino acids from the C-terminus of the protein. Furin cleavage occurs at the C-terminus of the cleavage site, which has the consensus sequence RXR(K)R, where X is any amino acid. In one aspect, the invention provides a means for removal of the newly exposed basic amino acid residues R or K from the C-terminus of the protein by use of an enzyme selected from a group of enzymes called carboxypeptidases (CPs), which include carboxypeptidase D, E and H (CPD, CPE, CPH). Since CPs are able to remove basic amino acid residues at the C-terminus of a protein, all amino acid resides derived from a furin cleavage site which contain exclusively basic amino acids R or K, such as RKKR (SEQ ID NO:18), RKRR (SEQ ID NO:19), RRKR (SEQ ID NO:20), RRRR (SEQ ID NO:21), etc, can be removed by a CP. A series of immunoglobulin expression constructs that contain a 2A sequence and a furin cleavage site and which have basic amino acid residues at the C terminus have been constructed to evaluate efficiency of cleavage and residue removal. An exemplary construct design is the following: H chain-furin (e.g., RKKR, RKRR, RRKR or RRRR)-2A-L chain or L chain-furin (e.g., RKKR, RKRR, RRKR or RRRR)-2A-H chain.

As will be apparent to those of skill in the art, there is a basic amino acid residue (K) at the C terminus of the immunoglobulin heavy (H) chain (rendering it subject to cleavage with carboxypeptidase), while the immunoglobulin light (L) chain, terminates with a non-basic amino acid C. In one preferred embodiment of the invention, an antibody expression construct comprising a furin sate and a 2A sequence is provided wherein the immunoglobulin L chain is 5′ to the immunoglobulin H chain such that following translation, the additional furin amino acid residues are cleaved with carboxypeptidase.

Recombinant AAV (rAAV) virions for use in practicing the present invention may be produced using standard methodology, known to those of skill in the art and are constructed such that they include, as operatively linked components in the direction of transcription, control sequences including transcriptional initiation and termination sequences, and the coding sequence for a therapeutic compound or biologically active fragment thereof. These components are bounded on the 5′ and 3′ end by functional AAV ITR sequences. By “functional AAV ITR sequences” is meant that the ITR sequences function as intended for the rescue, replication and packaging of the AAV virion. Hence, AAV ITRs for use in the vectors of the invention need not have a wild-type nucleotide sequence, and may be altered by the insertion, deletion or substitution of nucleotides or the AAV ITRs may be derived from any of several AAV serotypes. An AAV vector is a vector derived from an adeno-associated virus serotype, including without limitation, AAV-1, AAV-2, AAV-3, AAV-4, AAV-5, AAV-6, AAV-7, AAV-8, etc. Preferred AAV vectors have the wild type REP and CAP genes deleted in whole or part, but retain functional flanking ITR sequences. Table 2 illustrates exemplary AAV serotypes for use in gene transfer.

TABLE 2
AAV Serotypes For Use In Gene Transfer.

| | | Genome | Homology | |
|---|---|---|---|---|
| | | Size | vs | Immunity in |
| Serotype | Origin | (bp) | AAV2 | Human Population |
| AAV-1 | Human Specimen | 4718 | NT: 80% | NAB: 20% |
| | | | AA: 83% | |
| AAV-2 | Human Genital | 4681 | NT: 100% | NAB: 27-53% |
| | Abortion tissue | | AA: 100% | |
| | Amnion Fluid | | | |
| AAV-3 | Human | 4726 | NT: 82% | cross reactivity with |
| | Adenovirus | | AA: 88% | AAV2 NAB |
| | Specimen | | | |
| AAV-4 | African Green | 4774 | NT: 66% | Unknown |
| | Monkey | | AA: 60% | |
| AAV-5 | Human Genital | 4625 | NT: 65% | ELISA: 45% NAB: |
| | Lesion | | AA: 56% | 0% |
| AAV-6 | Laboratory isolate | 4683 | NT: 80% | 20% |
| | | | AA: 83% | |
| AAV-7 | Isolated from | 4721 | NT: 78% | NAB: <1:20 (˜5%) |
| | Heart DNA of | | AA: 82% | |
| | Rhesus Monkey | | | |
| AAV-8 | Isolated from | 4393 | NT: 79% | NAB: <1:20 (˜5%) |
| | Heart DNA of | | AA: 83% | |
| | Rhesus Monkey | | | |

Typically, an AAV expression vector is introduced into a producer cell, followed by introduction of an AAV helper construct, where the helper construct includes AAV coding regions capable of being expressed in the producer cell and which complement AAV helper functions absent in the AAV vector. The helper construct may be designed to down regulate the expression of the large REP proteins (Rep78 and Rep68), typically by mutating the start codon following p5 from ATG to ACG, as described in U.S. Pat. No. 6,548,286, expressly incorporated by reference herein. This is followed by introduction of helper virus and/or additional vectors into the producer cell, wherein the helper virus and/or additional vectors provide accessory functions capable of supporting efficient rAAV virus production. The producer cells are then cultured to produce rAAV. These steps are carried out using standard methodology. Replication-defective AAV virions encapsulating the recombinant AAV vectors of the instant invention are made by standard techniques known in the art using AAV packaging cells and packaging technology. Examples of these methods may be found, for example, in U.S. Pat. Nos. 5,436,146; 5,753,500, 6,040,183, 6,093,570 and 6,548,286, expressly incorporated by reference herein in their entirety. Further compositions and methods for packaging are described in Wang et al. (US 2002/0168342), also incorporated by reference herein in its entirety, and include those techniques within the knowledge of those of skill in the art.

Approximately 40 serotypes of AAV are currently known, however, new serotypes and variants of existing serotypes are still being identified today and are considered within the scope of the present invention. See Gao et al (2002), PNAS 99(18):11854-6; Gao et al (2003), PNAS 100(10):6081-6; Bossis and Chiorini (2003), J. Virol. 77(12):6799-810). Different AAV serotypes are used to optimize transduction of particular target cells or to target specific cell types within a particular target tissue, such as the brain. The use of different AAV serotypes may facilitate targeting of malignant tissue. AAV serotypes including 1, 2, 4, 5 and 6 have been shown to transduce brain tissue. See, e.g., Davidson et al (2000), PNAS 97(7)3428-32; Passini et al (2003), J. Virol 77(12):7034-40). Particular AAV serotypes may more efficiently target and/or replicate in target tissue or cells. A single self-complementary AAV vector can be used in practicing the invention in order to increase transduction efficiency and result in faster onset of transgene expression (McCarty et al., Gene Ther. 2001 August;8(16):1248-54).

In practicing the invention, immunoglobulin-encoding AAV constructs according to the present invention may be introduced into cells using standard techniques routinely employed by those of skill in the art.

Host cells can also be packaging cells in which the AAV REP and CAP genes are stably maintained in the host cell or alternatively host cells can be producer cells in which the AAV vector genome is stably maintained. Exemplary packaging and producer cells are derived from 293, A549 or HeLa cells. AAV vectors are purified and formulated using standard techniques routinely employed by those of skill in the art.

In practicing the invention, host cells for producing rAAV virions include mammalian cells, insect cells, microorganisms and yeast. For in vitro or ex vivo expression, any cell effective to express a functional immunoglobulin may be employed. Numerous examples of cells and cell lines used for protein expression are known in the art.

Examples of cells useful for immunoglobulin expression further include mammalian cells, such as fibroblast cells, cells from non-human mammals such as ovine, porcine, murine and bovine cells, insect cells and the like. Specific examples of mammalian cells include COS cells, VERO cells, HeLa cells, Chinese hamster ovary (CHO) cells, 293 cell, NSO cells, SP20 cells, 3T3 fibroblast cells, W138 cells, BHK cells, HEPG2 cells, DUX cells and MDCK cells.

Host cells are cultured in conventional nutrient media, modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium (MEM, Sigma), RPMI 1640 (Sigma), and Dulbecco's Modified Eagle's Medium (DMEM, Sigma) are typically suitable for culturing host cells. A given medium is generally supplemented as necessary with hormones and/or other growth factors (such as insulin, transferrin, or epidermal growth factor), DHFR, salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The appropriate culture conditions for a particular cell line, such as temperature, pH and the like, are generally known in the art, with suggested culture conditions for culture of numerous cell lines provided, for example, in the ATCC Catalogue available on line at <“http://www.atcc.org/ Search catalogs/AllCollections.cfm”>.

A vector encoding an immunoglobulin of the invention may be administered in vivo via any of a number of routes (e.g., intradermally, intravenously, intratumorally, into the brain, intraportally, intraperitoneally, intramuscularly, into the bladder etc.), effective to deliver the vector in animal models or human subjects. Dependent upon the route of administration, the immunoglobulin will elicit an effect locally or systemically. The use of a tissue specific promoter 5′ to the immunoglobulin open reading frame(s) results in greater tissue specificity with respect to expression of a recombinant protein expressed under control of a non-tissue specific promoter.

For example, in vivo delivery of the a recombinant AAV vector encoding a immunoglobulin of the invention may be targeted to a wide variety of organ types including, but not limited to brain, liver, blood vessels, muscle, heart, lung and skin. In vivo delivery of the recombinant AAV vector may also be targeted to a wide variety of cell types based on the serotype of the virus, the status of the cells, i.e. cancer cells may be targeted based on cell cycle, the hypoxic state of the cellular environment or other physiological status that deviates from the typical, or normal, physiological state of that same cell when in a non-cancerous (non-dividing or regulated dividing state under normal, physiological conditions).

In the case of ex vivo gene transfer, the target cells are removed from the host and genetically modified in the laboratory using a recombinant vector encoding an immunoglobulin according to the present invention and methods well known in the art.

The recombinant vectors of the invention can be administered using conventional modes of administration including but not limited to the modes described above and may be in a variety of formulations which include but are not limited to liquid solutions and suspensions, microvesicles, liposomes and injectable or infusible solutions. The preferred form depends upon the mode of administration and the therapeutic application.

Rapalog-Regulated Expression

A variety of expression systems have been developed, including regulated expression systems, which rely on switches triggered by a single drug such as tetracycline, RU486 or ecdysone, or on dimerization triggered by compounds such as a rapalog. One exemplary rapalog, rapamycin, is an orally bioavailable drug and thus finds utility in regulated gene expression in vivo as well as in vitro. Rapalog-regulated gene expression systems are described for example in U.S. Pat. Nos. 6,015,709; 6,117,680; 6,133,456; 6,150,527; 6,187,757; 6,306,649; 6,479,653 and 6,649,595, each of which is expressly incorporated by reference herein in it's entirety.

In one embodiment of the current invention, a modified version of ARIAD Regulation Technology is used which is based on the use of a small molecule to bring together two intracellular molecules, each of which is linked to either a transcriptional activator or a DNA binding protein. When these components come together, transcription of the immunoglobulin is activated. Two major systems which employ the ARIAD technology include a system based on homodimerization and a system based on heterodimerization (Rivera et al., 1996, Nature Med, 2(9):1028-1032; Ye et al., 2000, Science 283: 88-91; Rivera et al., PNAS, Vol. 96(15): 8657-8662, 1999).

In this system, the dimerizer inducible gene regulation system is comprised of 3 individual components: the activation domain, DNA binding domain, and the inducible promoter downstream of the antibody expression cassette of interest. In one exemplary embodiment, the activation domain is a fusion of the carboxy terminal from the p65 subunit of NF-kappa B and the large PI3K homolog FRAP domain (FRB), while the DNA binding domain is composed of a zinc finger pair from a transcription factor and a homeodomain joined to two copies of FK506 binding protein (FKBP). In the presence of an inducing agent, e.g., a rapalog such as rapamycin, the DNA binding domain and activation domain are dimerized through interaction of their FKBP and FRB domains, leading to transcription activation of the immunoglobulin gene. In one exemplary embodiment, the regulated promoter contains 8 binding sites followed by the minimal interleukin-2 (8×ZFHD1/IL-12) promoter. (See, e.g., Rivera, V. M., et al Blood, 2005. 105(4): p. 1424-30.)

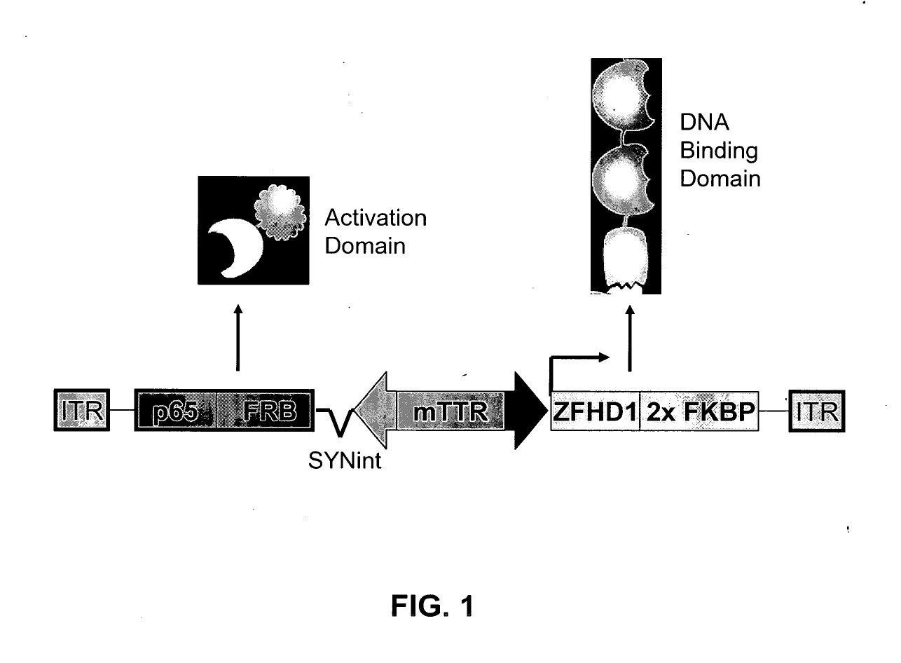

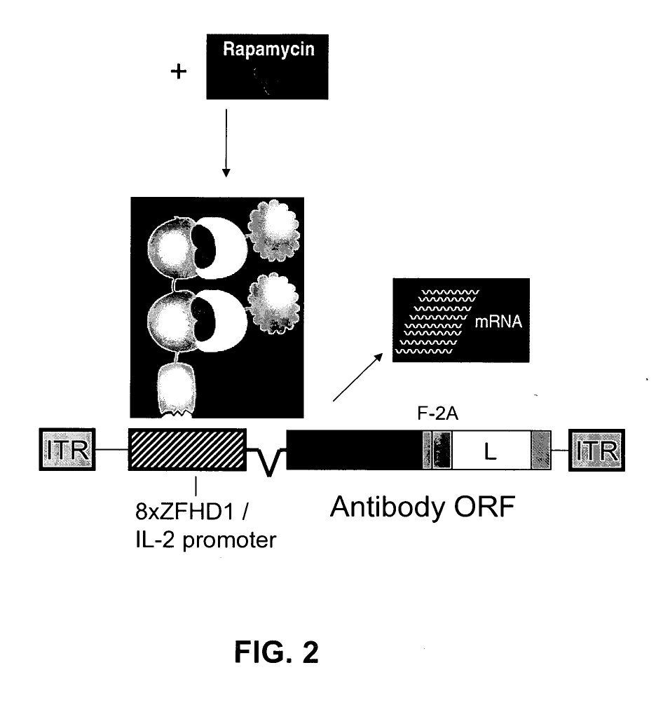

As described in the Examples, the three components of the rapalog regulation system have been cloned into two separate AAV vector constructs (FIGS. 1 & 2). The first AAV construct employs a bi-directional promoter comprised of the liver specific mouse transthyretin (TTR) promoter fused to the Simian virus 40 (SV40) minimal promoter, to express the activation protein (FRB-p65) and the DNA binding protein (ZFHD1-2×FKBP) respectively (FIG. 1). These two promoters share the TTR enhancer element allowing for liver-specific expression of FRB-p65 and ZFHD1-2×FKBP in opposite orientations. The second AAV construct comprises the rapamycin-regulated promoter 8×ZFHD1/IL-12 operably associated with the nucleotide sequence of the antibody expression cassette comprising a proteolytic cleavage site, a self-processing cleavage site (2A), and the nucleotide sequences encoding the immunoglobulin heavy (H) and light (L) chains (FIG. 2).

### EXAMPLES

### Example 1

### Construction of Rapamycin-Regulated AAV Vector Constructs

An AAV vector comprising the transactivation regulatory elements required for activating transcription from rapamycin-regulated promoters was constructed. AAV-CMV-TF1Nc (Auricchio, A., et al., Mol Ther, 2002. 6(2): p. 238-42), was used the source of AAV2 ITRs, the FRB-p65 activation domain and the ZFHD1-2×FKBP DNA binding domain. The IRES sequence was deleted and into this plasmid, the following elements were inserted: a mouse transthyretin mTTR promoter (Costa, R. H., et al., Mol Cell Biol, 1988. 8(1): p. 81-90) upstream of the FRB-p65 coding sequence, a synthetic intron: (5′gtatggatcctctaaaagcgggcatgacttctggggttgtcctgggtttccgtgggtttctaactgggccctttttttcacag-3′) (SEQ ID NO:15), between the mTTR promoter and FRB-p65 coding sequences, and a minimal human growth hormone poly adenylation signal hGHpA (5′-ccactccagtgcccaccagccttgtcctaataaaattaagttgcatcattttgtctgactaggtgtccttctataatattatggggtggagggggg tggmggagcaagg-3′) (SEQ ID NO:16), downstream of ZFHD1-2×FKBP. The ZFHD1-2×FKBP coding sequence is contained on a 1022 bp fragment was flanked upstream with the minimal SV40 promoter and downstream with a minimal rabbit beta globin polyA (5′-acgcctaataaagagctcagatgcatcgatcagagtgtgttggttttttgtgtgt-3′) (SEQ ID NO:22), and inserted in reverse orientation to the mTTR FRB-p65 cassette with the hGHpA relative to AAV-CMV-TF1Nc (FIG. 1).

A second AAV vector encoding full length heavy and light chains of a rat anti-FLK-1 monoclonal antibody with self processing cleavage sequences (2A) and a proteolytic cleavage site under the control of a rapamycin-regulated promoter was constructed (FIG. 2). The variable and constant regions of the antibody heavy and light chains were cloned from cDNA of the parental hydridoma cells using the Polymerase Chain Reaction (PCR). The cDNAs were synthesized with reverse transcriptase from total RNA isolated from the hydridoma cells using Qiagen's total RNA purification kit. The nucleotide sequences of the monoclonal antibodies were analyzed using an automatic sequencing system (Applied Biosystems) and consensus sequences were obtained from the sequencing data derived from multiple independent PCR reactions.

The DNA fragments that encode the heavy chain, 2A sequence and antibody light chain of either a rat mAb were linked together by PCR extension. Artificial FMDV 2A oligo nucleotides were synthesized based on the 2A peptide sequence APVKQTLNFDLLKLAGDVESNPGP (SEQ ID NO: 6). The heavy and light chain fragments were amplified from the cloned plasmids that encode the full-length antibody heavy and light chains respectively. During the PCR, an EcoR I restriction endonucleotidase site was added to the 5′ end of the heavy chain and the 3′ end of the light chain. The fused heavy chain-2A-light chain DNA fragment was digested with EcoR I and purified from agarose gel. The purified DNA fragment was inserted into an AAV plasmid backbone flanked with EcoR I sites using T4 DNA ligase. The proteolytic cleavage site, RAKR (SEQ ID NO:11), which belongs to the category of furin consensus cleavage sequences, was introduced into the 2A sequence between the self-processing site and the 3′-end of the heavy chain coding region. A 277 bp fragment containing the rapamycin-regulated promoter, 8×ZFHD1/IL-12, was isolated from AAV-Z12I-rhEpo2S6 (Auricchio, A., et al., Mol Ther, 2002. 6(2): p. 238-42) and inserted upstream of the immunoglobulin expression cassette in the same transcriptional orientation. A chimeric intron, pCI, was cloned between the rapamycin-regulated promoter and the nucleotide sequence encoding the heavy chain. In variant forms, a native signal peptide (leader) was included in the heavy or light chain, respectively, to facilitate secretion of the polypeptides upon synthesis.

The construct comprises in the 5′ to 3′ direction: a 5′ AAV ITR, the rapamycin-regulated promoter, the coding sequence for an antibody heavy chain (H), an additional proteolytic cleavage site coding sequence (e.g., a furin cleavage site coding sequence), the coding sequence for a self processing cleavage sequence (e.g., a 2A sequence), the coding sequence for an antibody light chain (L), and a polyA sequence (e.g., H-F2A-L). All constructs were fully sequenced using ABI PRISM 3100 (Applied Biosystems, Foster City, Calif.).

### Example 2

### Regulated Expression of DC101 Antibody from AAV Plasmids In Vitro

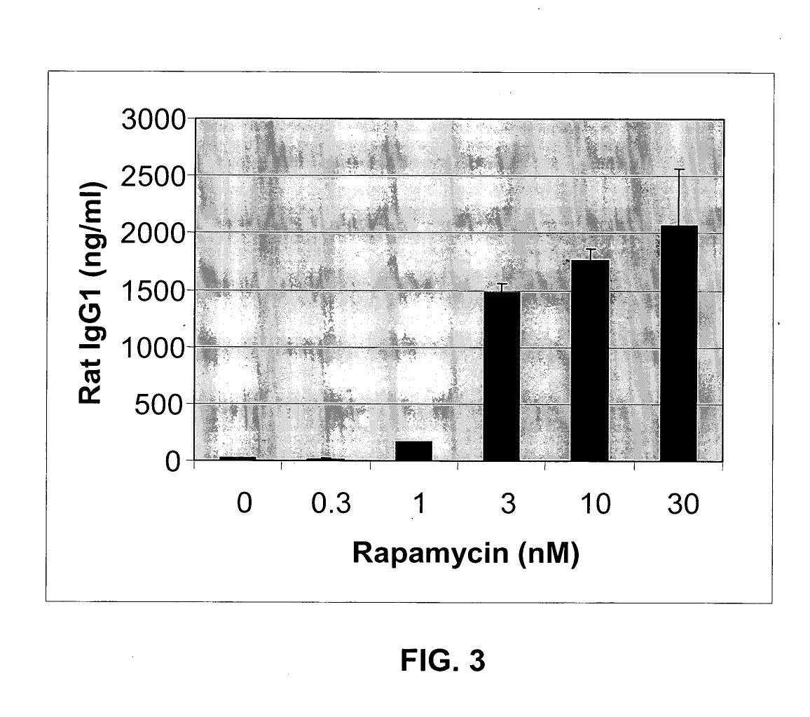

A monoclonal antibody was expressed from the regulated AAV plasmids in vitro, using HuH7 cells cultured in 6 well plates in the presence of increasing concentrations of rapamycin relative to a control which lacked rapamycin. The cells were transfected with the two AAV plasmids described in Example 1 by the FuGEN 6 transfection kit (Roche) following the manufacturer's instruction. After 24 hours, culture medium was replaced with fresh medium and the cells were returned into an incubator for additional culture for 48 hours. Then cell culture supernatants were collected and IgG1 was quantified using a rat IgG1 ELISA kit (Bethyl Laboratories). In the presence of Rapamycin, rat monoclonal antibody protein was detected in cell culture supernatants of HuH7 cells transfected by the plasmids (FIG. 3). In contrast, in the absence of rapamycin in culture medium, no antibody was detected in the supernatants from the cells that had been transfected by the two plasmids. In addition, there was no detectable rat antibody in the supernatants obtained from control wells that were not transfected by the antibody-encoding AAV plasmids

The results presented in FIG. 3 demonstrate that a full-length antibody can be expressed in vitro using co-transfection of the two AAV plasmids only in the presence of the inducer, rapamycin, wherein the antibody heavy and light chains are expressed as a single open reading frame using a self-processing sequence such as 2A. Furthermore, DC101 expression appears to be dependent on the concentration of rapamycin used for induction.

### Example 3

### AAV Production

In one example of the current invention, a regulated AAV vector is provided that produced high levels of biologically active antibody by use of a single promoter for expression of anti-FLK-1 antibody. Expression was accomplished using an antibody heavy chain-furin cleavage site-2A-light chain (H-F-2A-L) construct, allowing the antibody heavy and light chains to be expressed as a single open reading frame within the same cell. Pseudotyped rAAV serotype-8 vectors were produced in HEK 293 cells using calcium phosphate triple transfection of the rAAV vector expression plasmid in combination with the AAV-8 serotype helper plasmid p5e18-VD2/8 (Plate, K. H., et al., Nature, 1992. 359(6398): p. 845-8) and pXX-6 Galanis, E., et al., J Clin Oncol, 2005. 23(23): p. 5294-5304). Virions were isolated on two sequential CsCl gradients and titres determined by dot-blot using radioactive probe specific for the immunoglobulin genes.

### Example 4

### Regulation of Immunoglobluin Expression In Vivo

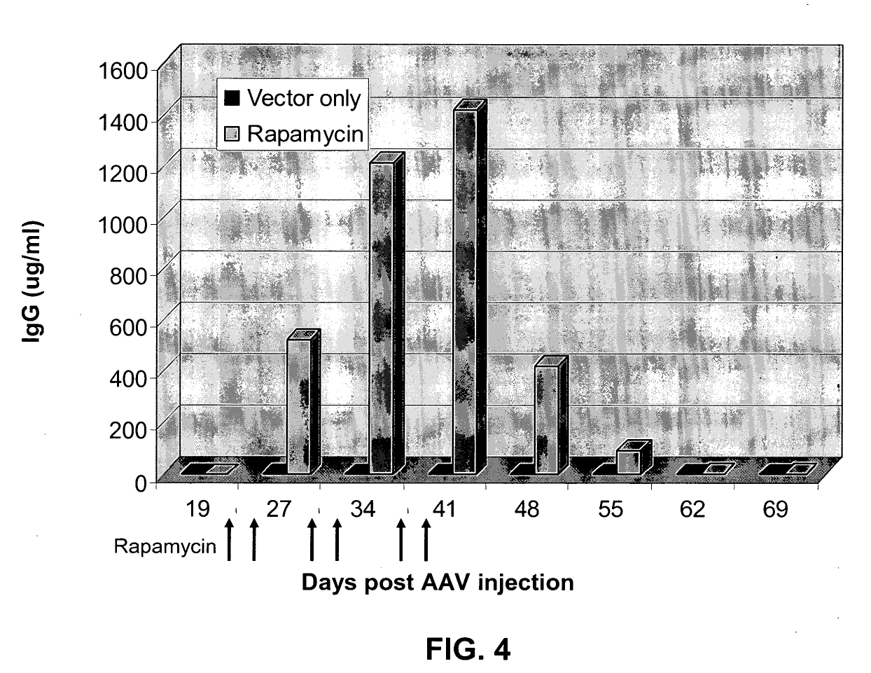

The regulated expression of a full-length rat anti-VEGFR2 monoclonal antibody (DC101 IgG1) from a rapamycin-regulated AAV vector construct in vivo is shown in FIG. 4. On Day 0, approximately 2.5×10¹¹ vp of each AAV vector described in Example were co-administered i.v. in the tail vein of six- to eight-week-old female NCR nu.nu nude mice. Mice were injected intraperitoneally with 40 ul of rapamycin—50% rapamycin stock of 3 mg/ml in DMA (Sigma, St. Louis, Mich.), 5% PEG-400 (Sigma), and 45% Tween-80 (Sigma)—to deliver a 3 mg/kg dose on Days 21, 24, 28, 31, 35, and 38. Mice were bled by alternate retro-orbital puncture on scheduled intervals to measure the serum levels and the amount of DC101 antibody present in serum samples (mcg/ml) at different time points was determined by an ELISA assay.

As shown in FIG. 4, DC101 expression was induced within one week after administration of rapamycin whereas virtually no expression was observed from control animals receiving only vector and vehicle. Multiple administrations of rapamycin further induced antibody expression and DC 101 levels exceeding 1 mg/ml were observed after 4 doses of rapamycin, e.g., Days 34 and 41. Measurable expression was detectable for about two weeks after the final administration of rapamycin (Day 55). These data demonstrate the tightly, regulated, inducible expression of a recombinant antibody in vivo.

TABLE 3
Brief Table of Sequences

| SEQ | |
|---|---|
| ID NO | SEQUENCE |
| 1 | LLNFDLLKLAGDVESNPGP |
| 2 | TLNFDLLKLAGDVESNPGP |
| 3 | LLKLAGDVESNPGP |
| 4 | NFDLLKLAGDVESNPGP |
| 5 | QLLNFDLLKLAGDVESNPGP |
| 6 | APVKQTLNFDLLKLAGDVESNPGP |
| 7 | VTELLYRMKRAETYCPRPLLAIHPTEARHKQKIVAPVKQTLNFD |
| | LLKLAGDVESNPGP |
| 8 | LLAIHPTEARHKQKIVAPVKQTLNFDLLKLAGDVESNPGP |
| 9 | EARHKQKIVAPVKQTLNFDLLKLAGDVESNPGP |
| 10 | RXK(R)R |
| 11 | RAKR |
| 12 | IE(D)GR |
| 13 | LAGFATVAQA |
| 14 | LVPRGS |
| 15 | gtatggatccctctaaaagcgggcatgacttctggggttgtcct |
| | gggtttccgtgggtttctactaactgggccctttttttttttca |
| | cag |
| 16 | ccactccagtgcccaccagccttgtcctaataaaattaagttgc |
| | atcattttgtctgactaggtgtccttctataatattatggggtg |
| | gaggggggtggtttggagcaagg |
| 17 | RXRKR |
| 18 | RKKR |
| 19 | RKRR |
| 20 | RRKR |
| 21 | RRRR |
| 22 | acgcctaataaagagctcagatgcatcgatcagagtgtgttggt |
| | tttttgtgtgt |

It is claimed:

1. An AAV vector for expression of a recombinant immunoglobulin, comprising:
in the 5′ to 3′ direction, a rapalog-regulated promoter operably linked to the coding sequence for a first chain of an immunoglobulin molecule or a fragment thereof, a proteolytic cleavage site, a sequence encoding a 2A self-processing cleavage site and the coding sequence for a second chain of an immunoglobulin molecule or a fragment thereof, wherein the sequence encoding the self-processing cleavage site is inserted between the coding sequence for the first chain and the coding sequence for the second chain of said immunoglobulin molecule.

2. An AAV vector according to claim 1, wherein said 2A sequence is a Foot and Mouth Disease Virus (FMDV) sequence.

3. An AAV vector according to claim 2, wherein the 2A sequence encodes a peptide comprising amino acid residues selected from the group consisting of the sequences presented as SEQ ID NO:1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9.

4. An AAV vector according to claim 3, wherein the 2A sequence encodes an oligopeptide comprising amino acid residues LLNFDLLKLAGDVESNPGP (SEQ ID NO:1) or TLNFDLLKLAGDVESNPGP (SEQ ID NO:2).

5. A vector according to claim 3, wherein the 2A sequence encodes an oligopeptide comprising amino acid residues APVKQTLNFDLLKLAGDVESNPGP (SEQ ID NO: 6).

6. An AAV vector according to claim 1, wherein the coding sequence for the first chain of said immunoglobulin molecule or a fragment thereof encodes an immunoglobulin heavy chain.

7. An AAV vector according to claim 1, wherein the coding sequence for the first chain of said immunoglobulin molecule or a fragment thereof encodes an immunoglobulin light chain.

8. An AAV vector according to claim 6, wherein the coding sequence is the full length coding sequence of an immunoglobulin heavy chain.

9. An AAV vector according to claim 7, wherein the coding sequence is the full length coding sequence of an immunoglobulin light chain.

10. An AAV vector according to claim 1, wherein said proteolytic cleavage site is a furin cleavage site with the consensus sequence RXK(R)R (SEQ ID NO:10).

11. An AAV vector according to claim 1, wherein said heavy and light chain immunoglobulin coding sequences are expressed in an equimolar ratio.

12. An AAV vector according to claim 4, wherein said heavy and light chain immunoglobulin coding sequences are expressed in an equimolar ratio.

13. An AAV vector according to claim 1, wherein said heavy and light chain immunoglobulin coding sequences are expressed in an equimolar ratio.

14. An AAV vector according to claim 1, further comprising a signal sequence.

15. An AAV vector according to claim 1, wherein vector said AAV vector is an AAV6 vector.

16. An AAV vector according to claim 1, wherein vector said AAV vector is an AAV8 vector.

17. A recombinant immunoglobulin molecule produced by a cell transduced with a vector of claim 10.

18. A host cell transduced with a vector of claim 10.

19. A recombinant immunoglobulin molecule produced by a cell transduced with a vector of claim 12.

20. A host cell transduced with a vector of claim 12.

21. A recombinant immunoglobulin molecule produced by a cell transduced with a vector of claim 13.

22. A host cell transduced with a vector of claim 13.

23. A method for producing a recombinant immunoglobulin molecule, comprising the steps of:
a. transducing a host cell with a vector according to claim 1; and
b. expressing said recombinant immunoglobulin in said transduced host cell, wherein said first immunoglobulin coding sequence and said second immunoglobulin coding sequence are expressed in a substantially equimolar ratio.

24. The method according to claim 23, wherein said 2A sequence is a Foot and Mouth Disease Virus (FMDV) sequence.

25. The method according to claim 23, wherein the 2A sequence encodes a peptide comprising amino acid residues LLNFDLLKLAGDVESNPGP (SEQ ID NO:1) or TLNFDLLKLAGDVESNPGP (SEQ ID NO:2).

26. The method according to claim 23, wherein the 2A sequence encodes a peptide comprising amino acid residues APVKQTLNFDLLKLAGDVESNPGP (SEQ ID NO: 6).

27. The method according to claim 23, wherein said additional proteolytic cleavage site is a furin cleavage site with the consensus sequence RXK(R)R (SEQ ID NO:10).

28. The method according to claim 23, further comprising treating said expressed immunoblobulin with a carboxypeptidase.

29. A system for regulated expression of a recombinant immunoglobulin from a single cell, comprising:
an AAV vector according to claim 1, and a host cell transduced with said vector, wherein said first immunoglobulin coding sequence and said second immunoglobulin coding sequence are expressed in a substantially equimolar ratio.

30. The system according to claim 29, wherein the 2A sequence encodes a peptide comprising amino acid residues LLNFDLLKLAGDVESNPGP (SEQ ID NO:1) or TLNFDLLKLAGDVESNPGP (SEQ ID NO:2).

31. The system according to claim 29, wherein the 2A sequence encodes a peptide comprising amino acid residues APVKQTLNFDLLKLAGDVESNPGP (SEQ ID NO: 6).

32. The system according to claim 29, wherein said additional proteolytic cleavage site is a furin cleavage site with the consensus sequence RXK(R)R (SEQ ID NO:10).


SEQUENCE LISTING (22 sequences)

SEQ ID NO: 1 - protein, 19 aa, Foot-and-mouth disease virus
LLNFDLLKLAGDVESNPGP

SEQ ID NO: 2 - protein, 19 aa, Foot-and-mouth disease virus
TLNFDLLKLAGDVESNPGP

SEQ ID NO: 3 - protein, 14 aa, Foot-and-mouth disease virus
LLKLAGDVESNPGP

SEQ ID NO: 4 - protein, 17 aa, Foot-and-mouth disease virus
NFDLLKLAGDVESNPGP

SEQ ID NO: 5 - protein, 20 aa, Foot-and-mouth disease virus
QLLNFDLLKLAGDVESNPGP

SEQ ID NO: 6 - protein, 24 aa, Foot-and-mouth disease virus
APVKQTLNFDLLKLAGDVESNPGP

SEQ ID NO: 7 - protein, 58 aa, Foot-and-mouth disease virus
VTELLYRMKRAETYCPRPLLAIHPTEARHKQKIVAPVKQTLNFDLLKLAGDVESNPGP

SEQ ID NO: 8 - protein, 40 aa, Foot-and-mouth disease virus
LLAIHPTEARHKQKIVAPVKQTLNFDLLKLAGDVESNPGP

SEQ ID NO: 9 - protein, 33 aa, Foot-and-mouth disease virus
EARHKQKIVAPVKQTLNFDLLKLAGDVESNPGP

SEQ ID NO: 10 - protein, 5 aa, Artificial Sequence
  Description of Artificial Sequence Synthetic peptide
  MOD_RES (2): Variable amino acid
RXKRR

SEQ ID NO: 11 - protein, 4 aa, Artificial Sequence
  Description of Artificial Sequence Synthetic peptide
RAKR

SEQ ID NO: 12 - protein, 5 aa, Artificial Sequence
  Description of Artificial Sequence Synthetic peptide
IEDGR

SEQ ID NO: 13 - protein, 10 aa, Artificial Sequence
  Description of Artificial Sequence Synthetic peptide
LAGFATVAQA

SEQ ID NO: 14 - protein, 6 aa, Artificial Sequence
  Description of Artificial Sequence Synthetic peptide
LVPRGS

SEQ ID NO: 15 - DNA, 91 bp, Artificial Sequence
  Description of Artificial Sequence Synthetic oligonucleotide
gtatggatccctctaaaagcgggcatgacttctggggttgtcctgggtttccgtgggtttctactaactgggccctttttttttttcacag

SEQ ID NO: 16 - DNA, 111 bp, Artificial Sequence
  Description of Artificial Sequence Synthetic oligonucleotide
ccactccagtgcccaccagccttgtcctaataaaattaagttgcatcattttgtctgactaggtgtccttctataatattatggggtggaggggggtggtttggagcaagg

SEQ ID NO: 17 - protein, 5 aa, Artificial Sequence
  Description of Artificial Sequence Synthetic peptide
  MOD_RES (2): Variable amino acid
RXRKR

SEQ ID NO: 18 - protein, 4 aa, Artificial Sequence
  Description of Artificial Sequence Synthetic peptide
RKKR

SEQ ID NO: 19 - protein, 4 aa, Artificial Sequence
  Description of Artificial Sequence Synthetic peptide
RKRR

SEQ ID NO: 20 - protein, 4 aa, Artificial Sequence
  Description of Artificial Sequence Synthetic peptide
RRKR

SEQ ID NO: 21 - protein, 4 aa, Artificial Sequence
  Description of Artificial Sequence Synthetic peptide
RRRR

SEQ ID NO: 22 - DNA, 55 bp, Artificial Sequence
  Description of Artificial Sequence Synthetic oligonucleotide
acgcctaataaagagctcagatgcatcgatcagagtgtgttggttttttgtgtgt